(19) United States
(12) Patent Application Publication
(10) Pub. No.: US 2022/0299580 A1
(43) Pub. Date: Sep. 22, 2022

(54) MAGNETIC-FIELD MEASURING APPARATUS

(71) Applicants: Takashi YASUI, Tokyo (JP); Daisuke OYAMA, Ishikawa (JP)

(72) Inventors: Takashi YASUI, Tokyo (JP); Daisuke OYAMA, Ishikawa (JP)

(73) Assignees: Ricoh Company, Ltd., Tokyo (JP); KANAZAWA INSTITUTE OF TECHNOLOGY, Ishikawa (JP)

(21) Appl. No.: 17/753,268

(22) PCT Filed: Sep. 24, 2020
(86) PCT No.: PCT/JP2020/036142

§ 371 Date: Feb. 25, 2022

(30) Foreign Application Priority Data
Oct. 2, 2019 (JP) 2019-182193
Oct. 11, 2019 (JP) 2019-188211

(51) Int. Cl. G01R 33/035; A61B 5/00

(52) U.S. Cl. CPC G01R 33/0356; A61B 5/0046; A61B 2562/0223; A61B 2562/182

(57) ABSTRACT

A magnetic-field measuring apparatus includes a SQUID; and flux-locked loop circuitry including first circuitry that includes an amplifier connected to an output of the SQUID, and second circuitry connected to the first circuitry. The first circuitry is along an inner surface or an outer surface of a shielding material that separates an inside of a magnetically shielded room from an outside of the magnetically shielded room, the magnetically shielded room including the SQUID. The second circuitry is in the outside of the magnetically shielded room.

### TECHNICAL FIELD

The present invention relates to a magnetic-field measuring apparatus.

### BACKGROUND ART

Measurement of a weak magnetic field, such as magnetism of a living body, is implemented typically using superconducting quantum interference devices (SQUIDs) in a magnetically shielded room (MSR). Hereinafter, a superconducting quantum interference device will be also referred to as a SQUID. SQUIDs have non-linear responses to magnetic fields, and the non-linear responses are linearized using flux-locked loop (FLL) circuitry to measure the magnetic fields. As FLL circuitry, an analog FLL system, which includes exclusively analog circuitry, and a digital FLL system, which converts analog signals into digital signals and converts the digital signals back into analog signals after signal processing, are known.

Magnetic-field signals measured by SQUIDs are weak and susceptible to noise. Therefore, a method for dividing control circuitry for controlling operation of SQUIDs into two sections is disclosed. The one of the two sections is installed in a magnetically shielded room and the other is installed outside the magnetically shielded room (see PTL 1).

For example, in a case where a cooling system including SQUIDs and headsets including preamplifiers are directly connected, the distance between the SQUIDs and the preamplifiers can be minimized, thereby enabling to improve the noise robustness with respect to the signals that are output from the SQUIDs to be amplified by the preamplifiers. However, long cables are used to connect the preamplifiers with control circuitry that is outside the magnetically shielded room. In particular, longer cables are used in the magnetically shielded room than the corresponding cables of a case where the preamplifiers are provided away from the cooling system.

The cables connecting the preamplifiers and the control circuitry include not only signal cables for transmitting the signals amplified by the preamplifiers but also power cables for supplying power to the preamplifiers. Therefore, if the longer cables are used in the magnetically shielded room, greater radiant magnetic field noise is generated in the magnetically shielded room, and thus, greater radiant magnetic field noise is detected by the SQUIDs. Therefore, even if the noise robustness with respect to the signals before being amplified by the preamplifiers is improved, the pream-plifiers amplify the radiant magnetic field noise detected by the SQUIDs together with the biomagnetic field signals.

### SUMMARY OF INVENTION

### Technical Problem

An aspect of the present invention has been devised in view of the above-described problem and is intended to provide a magnetic-field measuring apparatus capable of reducing radiant magnetic field noise generated in a magnetically shielded room.

### Solution to Problem

In order to solve the above described problem, a magnetic-field measuring apparatus according to the aspect of the present invention includes a SQUID; and flux-locked loop circuitry including first circuitry that includes an amplifier connected to an output of the SQUID, and second circuitry connected to the first circuitry. The first circuitry is along an inner surface or an outer surface of a shielding material that separates an inside of a magnetically shielded room from an outside of the magnetically shielded room, the magnetically shielded room including the SQUID. The second circuitry is in the outside of the magnetically shielded room.

Thus, the magnetic-field measuring apparatus capable of reducing radiant magnetic field noise generated in a magnetically shielded room can be provided.

Other objects, features and advantages of the present invention will become more apparent from the following detailed description when read in conjunction with the ac-companying drawings.

### BRIEF DESCRIPTION OF DRAWINGS

FIG. 1 is a block diagram illustrating an example of a magnetic-field measuring apparatus according to a first embodiment of the present invention.

FIG. 2A is a layout diagram illustrating an example of installation of the magnetic-field measuring apparatus (magnetoencephalograph) of FIG. 1.

FIG. 2B is an enlarged partial cross-sectional view of a variation in materials of a wall of a magnetically shielded room.

FIG. 3 is a layout diagram illustrating another example of installation of the magnetic-field measuring apparatus (magnetospinograph) of FIG. 1.

FIG. 4 is a block diagram illustrating an example of a magnetic-field measuring apparatus according to a second embodiment of the present invention.

FIG. 5 is a layout diagram illustrating an example of installation of the magnetic-field measuring apparatus (magnetoencephalograph) of FIG. 4.

FIG. 6 is a layout diagram illustrating another example of installation of the magnetic-field measuring apparatus (magnetospinograph) of FIG. 4.

FIG. 7 is an explanatory diagram illustrating examples of wiring routes in the magnetic-field measuring apparatus of FIG. 1.

FIG. 8 is an explanatory diagram illustrating examples of wiring routes in the magnetic-field measuring apparatuses of FIGS. 1 and 5.

FIG. 9 depicts frequency spectrums for illustrating measurement results of magnetic field noise with respect to wiring routes WR1 and WR2 illustrated in FIG. 7.

FIG. 10 depicts frequency spectrums for illustrating measurement results of magnetic field noise with respect to wiring routes WR3 and WR4 illustrated in FIG. 7.

FIG. 11 depicts frequency spectrums for illustrating measurement results of magnetic field noise with respect to wiring routes WR5 and WR6 illustrated in FIG. 8.

FIG. 12 depicts frequency spectrums for illustrating a measurement result of magnetic field noise with respect to a wiring route WR7 illustrated in FIG. 8.

FIG. 13 depicts frequency spectrums for illustrating an example of a magnetic-field measuring apparatus according to a third embodiment of the present invention.

FIG. 14 is a layout diagram illustrating an example (comparative example) of installation of another magnetic-field measuring apparatus.

FIG. 15 is a layout diagram illustrating another example (comparative example) of installation of the magnetic-field measuring apparatus.

### DESCRIPTION OF EMBODIMENTS

Now, embodiments will be described with reference to the drawings. In each drawing, the same elements are indicated by the same reference numerals and duplicate descriptions may be omitted.

### First Embodiment

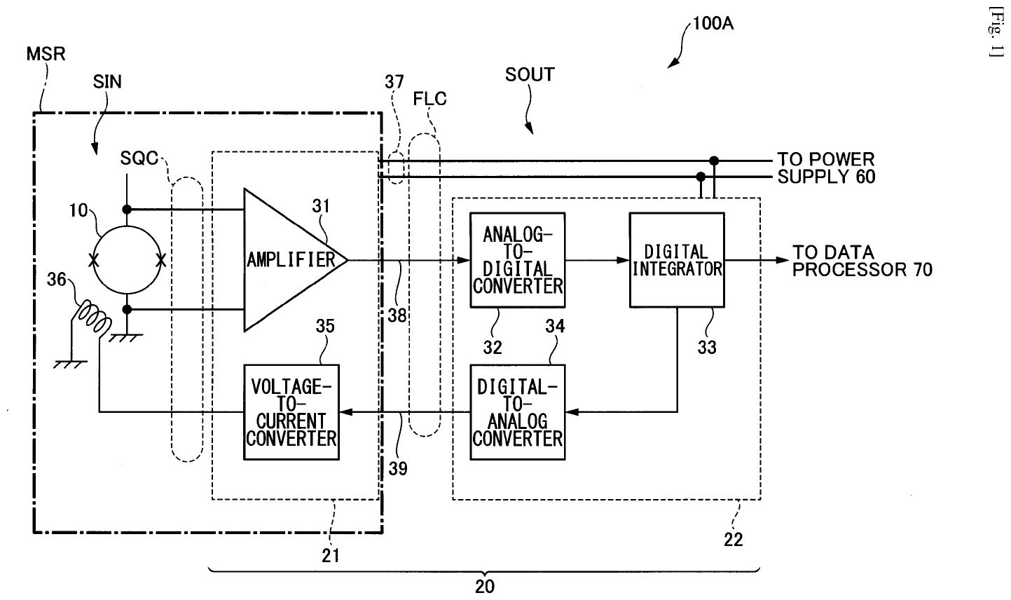

FIG. 1 is a block diagram illustrating an example of a magnetic-field measuring apparatus according to a first embodiment of the present invention. For example, the magnetic-field measuring apparatus 100A illustrated in FIG. 1 uses a digital FLL method and can be applied to a magnetoencephalograph (MEG), a magnetospinograph (MSG), or a magnetocardiograph (MCG). The magnetic-field measuring apparatus 100A illustrated in FIG. 1 may also be applied to a magnetomyograph.

The magnetic-field measuring apparatus 100A includes a SQUID 10, a digital FLL circuitry 20, and a feedback coil 36. The SQUID 10 is a highly sensitive magnetic sensor that detects a magnetic field (magnetic flux), generated from a living body, passing through a superconducting ring having Josephson junctions. For example, in the SQUID 10, Josephson junctions are provided at two positions of the super-conducting ring.

The SQUID 10 generates a voltage that varies periodically with a change in a magnetic flux passing through the superconducting ring. Therefore, a magnetic flux passing through the superconducting ring can be detected by measuring a voltage across the superconducting ring with a bias current applied to the superconducting ring. Hereinafter, the characteristic of a periodic voltage change generated by the SQUID 10 is also referred to as a Φ-V characteristic.

The digital FLL circuitry 20 includes an amplifier 31, an analog-to-digital converter 32, a digital integrator 33, a digital-to-analog converter 34, and a voltage-to-current converter 35. In the digital FLL circuitry 20, the amplifier 31 and the voltage-to-current converter 35 are included in first circuitry 21; and the analog-to-digital converter 32, the digital integrator 33 and the digital-to-analog converter 34 are included in second circuitry 22. A feedback coil 36 near the SQUID 10 is physically apart from the digital FLL circuitry 20, but may be included in the functional block of the digital FLL circuitry 20.

A plurality of electrical cables FLC, such as power supply cables 37 and control signal lines (an amplifier output line 38 and a voltage line 39) are installed between the first circuitry 21 and the second circuitry 22. A plurality of electrical cables SQC are installed to connect the SQUID 10 and the feedback coil 36 with the first circuitry 21. Hereinafter, the electrical cables SQC and FLC are also simply referred to as cables SQC and FLC.

In the present embodiment, the first circuitry 21, which does not include digital circuitry, is located in the magnetically shielded room MSR, together with the SQUID 10 and the feedback coil 36. The second circuitry 22, including digital circuitry, is located outside the magnetically shielded room MSR. Therefore, it is possible to prevent radiant magnetic field noise, generated by operation of digital circuitry operating in synchronization with a clock signal, for example, from being transmitted in the magnetically shielded room MSR. This prevents the SQUID 10 from detecting radiant magnetic field noise generated by digital circuitry.

Further, because the first circuitry 21 includes only minimum circuitry, the volume of the first circuitry 21 housed by the magnetically shielded room MSR can be minimized, and the magnetically shielded room MSR can be effectively used.

Herein, the term “magnetically shielded room” means a space formed by using, as a shielding material (wall material), a magnetic material or a material having a high electrical conductivity alone or in a lamination to reduce an influence of environmental magnetic field noise on the inside.

A magnetic field emitted from a living body is very weak, ranging from 10⁻¹⁰ to 10⁻¹⁴ T (tesla). Therefore, in order to accurately detect a magnetic field using the SQUID 10, it is desired to reduce urban noise (environmental magnetic field noise). Therefore, the magnetically shielded room is required to reduce environmental magnetic field noise by approximately 60 dB. Hereinafter, an example of the magnetically shielded room will be described, but there is no limitation to the example.

The magnetically shielded room MSR is formed by covering an internal space SIN, in which the SQUID 10 is installed, with a shielding member (indicated by an alternate long and short dash line) that shields magnetism. The shielding member corresponds to a shielding material (wall material) that separates the inside of the magnetically shielded room MSR from the outside of the magnetically shielded room MSR. As a result, the internal space SIN which is the inside of the magnetically shielded room MSR in which the SQUID 10 is installed is magnetically shielded from an external space SOUT which is the outside of the magnetically shielded room MSR. By placing the SQUID 10 in the magnetically shielded room MSR, it is possible to reduce an influence of an environmental magnetic field such as a geomagnetic field.

The amplifier 31 amplifies an output voltage, generated by the SQUID 10 from a magnetic flux passing through the SQUID 10, to output the amplified output voltage to the analog-to-digital converter 32 via an amplifier output line 38. The analog-to-digital converter 32 converts the analog signal output by the amplifier 31 into a digital signal (voltage value), by sampling the analog signal at a predetermined sampling frequency, to output the digital signal to the digital integrator 33.

The digital integrator 33 integrates a change in the voltage of the SQUID 10 (precisely, the amplified voltage output from the amplifier 31) from a working point, which is a starting point of each cycle with respect to the Φ-V characteristic, and outputs the integrated voltage value to the digital-to-analog converter 34.

The digital integrator 33 outputs the integrated voltage value to a data processor 70 such as a computer. The data processor 70 calculates a value of a biomagnetic field signal, which is a magnetic flux emitted by a living body (a brain, a heart, a nerve, or the like) to be measured, based on the voltage value from the digital integrator 33.

The digital-to-analog converter 34 converts the integrated voltage value (the digital signal) of the digital integrator 33 to a voltage and outputs the converted voltage to the voltage-to-current converter 35 via the voltage line 39. The voltage-to-current converter 35 converts the voltage received from the digital-to-analog converter 34 into a current and outputs the converted current to the feedback coil 36.

The feedback coil 36 generates a magnetic flux due to the current received from the voltage-to-current converter 35 and feeds the generated magnetic flux back to the SQUID 10 as a feedback magnetic flux. As a result, the voltage generated by the SQUID 10 can be maintained near the working point (linear zone) with respect to the Φ-V characteristic, and thus, the biomagnetic field signal can be accurately obtained.

The configuration illustrated in FIG. 1 corresponds to one channel of the magnetic-field measuring apparatus 100A. One channel includes a corresponding one SQUID 10 and corresponding digital FLL circuitry 20 connected to the SQUID 10. Hereinafter, the magnetic-field measuring apparatus 100A will be described as having 128 channels. In this regard, the magnetic-field measuring apparatus 100A may have 200 channels or more.

In the following description, it is assumed that, for example, eight cables SQC are installed for each channel from the SQUID 10 and the feedback coil 36 to the first circuitry 21, and eight cables FLC are installed for each channel between the first circuitry 21 and the second circuitry 22. That is, the magnetic-field measuring apparatus 100A with the 128 channels has 1024 cables SQC and 1024 cables FLC. The cables SQC and FLC include cables for control in addition to the cables illustrated in FIG. 1. Also, power cables from among the cables FLC may be shared by a predetermined number of channels.

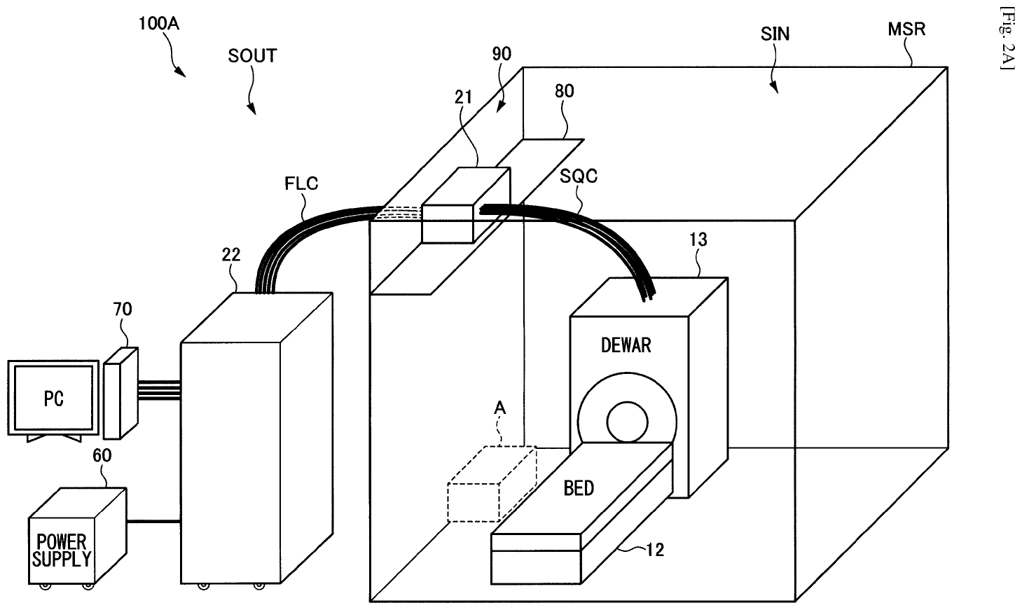

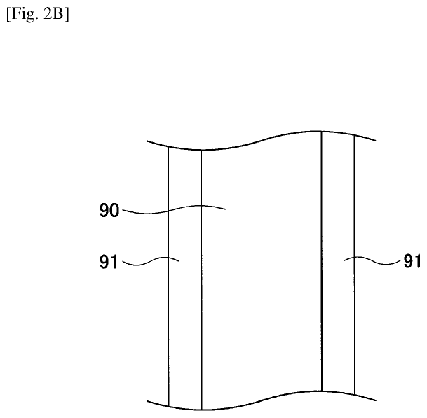

FIG. 2A is a layout diagram illustrating an example of installation of the magnetic-field measuring apparatus 100A of FIG. 1. For example, the magnetic-field measuring apparatus 100A illustrated in FIG. 2A is a magnetoencephalograph with a bed 12 on which a subject lies and a dewar 13 (cryogenic container) having a depression in which the head of the subject lying on the bed is located approximately at the center of the magnetically shielded room MSR. The dewar 13 is a container providing a cryogenic environment using liquid helium, and plural sets of SQUIDs 10 and feedback coils 36 for magnetoencephalography are disposed in the inside of the dewar 13 under the depression.

With regard to the magnetically shielded room MSR, the shielding material 90 (wall material) that separates the internal space SIN from the external space SOUT is formed by laminating of, for example, a plate material made of permalloy or the like as a high magnetic permeability material and a plate material made of an electrical conductor such as copper or aluminum. The magnetically shielded room MSR, as illustrated in FIG. 1, generally has a rectangular-parallelepiped shape and the four side walls, ceiling, and floor are formed of the shielding material 90. The magnetically shielded room MSR has a door that enables various devices to be transported and people to enter the magnetically shielded room. However, indication of the door is omitted in FIG. 2A and also in the following figures.

For example, the first circuitry 21 is installed on a shelf 80 installed at an upper section of an inner wall (inner surface) of the shielding material 90 of the magnetically shielded room MSR obliquely above the dewar 13. That is, the first circuitry 21 is located at the upper section of the inner wall of the magnetically shielded room MSR and higher than the top surface of the dewar 13.

The internal space SIN of the magnetically shielded room MSR can be effectively used by installing of the first circuitry 21 on the shelf. In addition, by placing the first circuitry 21 at a higher position than the top surface of the dewar 13, the cables SQC can be introduced to a cable insertion opening of the dewar 13 due to gravity, thereby reducing the influence of the load of the cables SQC applied to the cable insertion opening.

The plurality of cables SQC are installed to connect the first circuitry 21 with the SQUIDs 10 and the feedback coils 36 (not illustrated) disposed inside the dewar 13. For example, the cables SQC are inserted into the dewar 13 through the cable insertion opening formed at the top of the dewar 13.

In addition, the first circuitry 21 and the second circuitry 22 installed in the external space SOUT of the magnetically shielded room MSR are connected to each other through the cables FLC which pass through a through hole provided in a wall of the magnetically shielded room MSR. The first circuitry 21 may be placed on the floor surface in contact with or near the inner wall of the magnetically shielded room MSR. However, in order to prevent the cables FLC from being exposed to the internal space SIN and to prevent the radiant magnetic field noise generated from the cables FLC from being radiated to the internal space SIN, it is desirable that the first circuitry 21 be in contact with the inner wall (see FIG. 2A).

In this regard, the shielding material 90 may be protected by protection materials 91 as depicted in FIG. 2B. The protection materials may be gypsum boards, wooden boards, polyurethane foam materials, or the like. In such a case, instead of the first circuitry 21 being directly in contact with (the inner wall of) the shielding material 90, the first circuitry may be in contact with (the inner wall of) the shielding material 90 via the protection material 91. Such a variation in the relationship between the first circuitry 21 and the shielding material 90 may also apply to all of the other examples described herein.

With regard to the cables FLC connecting the first circuitry 21 and the second circuitry 22, the length of the cables FLC from the first circuitry 21 to the inner surface of the shielding material 90 is smaller than the length of the cables SQC connecting the SQUIDs 10 and the feedback coils 36 with the first circuitry 21. That is, the length of the cables FLC from the first circuitry 21 to the inner surface of the shielding material 90 is smaller than the length of the cables SQC from the dewar 13 to the first circuitry 21.

As to the cables SQC and FLC, for example, 136-core cord-assembled-type cables are used. Hereinafter, a cable in which 136 cables SQC (or FLC) are assembled will also be referred to as cables SQC (or FLC). With regard to the 1024 cables SQC and 1024 cables FLC, the number of cord-assembled-type cables SQC and the number of cord-assembled-type cables FLC used in the internal space SIN and the external space SOUT is eight each.

The cord-assembled-type cables SQC and FLC are thicker and have greater flexural rigidity than individual cables SQC and FLC. Therefore, for example, it is desirable that the first circuitry 21 be positioned so as to minimize the bending of the cables SQC and prevent the load of the cables SQC from concentrating at the cable insertion opening of the dewar 13. In this regard, the first circuitry 21 is desirably located obliquely above the dewar 13.

For example, the first circuitry 21 is provided in a form of at least one printed circuit board housed in a housing illustrated as having a rectangular parallelepiped shape in FIG. 2A. In this case, the housing is desirably formed of a material capable of shielding radiant magnetic field noise generated by the power supply lines installed on the printed circuit board. The first circuitry 21 may include the housing illustrated as having the rectangular parallelepiped shape in FIG. 2A and at least one printed circuit board housed by the housing. Hereinafter, the housing in which the first circuitry 21 is housed is also included when referring to the first circuitry 21.

In the following description, the state where the first circuitry 21 is in contact with or near an inner wall or the like means a state where a part of the first circuitry 21 facing the inner wall or the like is in contact with or near the inner wall or the like. For example, when the first circuitry 21 includes the rectangular parallelepiped housing, the state where the first circuitry 21 is in contact with or near an inner wall or the like means a state where a side, top or bottom surface of the housing facing the inner wall or the like is in contact with or near the inner wall or the like.

The second circuitry 22 is provided in a form of at least one printed circuit board housed in a housing such as a rack illustrated as having a rectangular parallelepiped shape in FIG. 2A. Hereinafter, the housing in which the second circuitry 22 is housed is also included when referring to the second circuitry 22.

In the present embodiment, because the first circuitry 21 is installed along the inner wall of the shielding material 90 of the magnetically shielded room MSR, it is possible to prevent the cables FLC including the power supply cables 37 (see FIG. 1) from being installed in the magnetically shielded room MSR. Accordingly, the SQUIDs 10 disposed in the dewar 13 can be prevented from detecting radiant magnetic field noise generated from the power supply cables 37, thereby improving the accuracy of the measurement of biomagnetic field signals by the SQUIDs 10.

The first circuitry 21 may be installed directly to the inner wall of the magnetically shielded room MSR above the dewar 13 or the first circuitry 21 may be installed to the inner wall via a mounting panel or the like. As indicated by the dashed-line box A in FIG. 2, the first circuitry 21 may be placed on the floor surface in contact with or near the inner wall of the magnetically shielded room MSR. In this regard, as described above, it is desirable that the first circuitry 21 be installed in contact with the inner wall. By placing the first circuitry 21 on the floor surface, the load of the cables SQC can be partially received by the floor to reduce the influence of the load of the cables SQC applied to the cable insertion opening of the dewar 13.

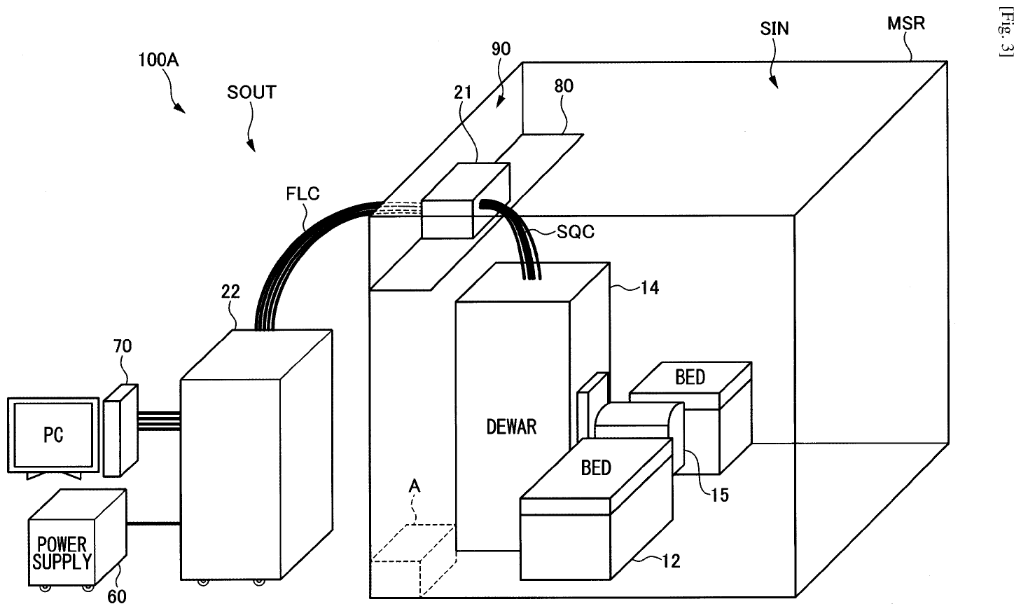

FIG. 3 is a layout diagram illustrating another example of installation of the magnetic-field measuring apparatus 100A of FIG. 1. For the same elements as the elements illustrated in FIG. 2A, the same numerals are used, and the detailed description will be omitted.

For example, the magnetic-field measuring apparatus 100A illustrated in FIG. 3 is a magnetospinograph. Approximately at the center of the magnetically shielded room MSR, a split bed 12, where the subject lies in a supine body position, and a case 15 of the SQUIDs 10 at a position corresponding to the subject's lower back, who lies on the bed 12, are installed. The SQUIDs 10 and the feedback coils 36 illustrated in FIG. 1 are disposed within the case 15.

Similar to FIG. 2A, the first circuitry 21 is positioned on the shelf 80, installed to an inner wall of the shielding material 90 of the magnetically shielded room MSR, above a dewar 14. That is, the first circuitry 21 is located at an upper section of the inner wall of the magnetically shielded room MSR higher than the top surface of the dewar 14. Thus, the internal space SIN of the magnetically shielded room MSR can be effectively used in the same manner as the example of FIG. 2A. In addition, the cables SQC can be introduced to the cable insertion opening of the dewar 14 due to gravity, thereby reducing the load of the cables SQC applied to the cable insertion opening.

The first circuitry 21 may be installed directly to the inner wall of the magnetically shielded room MSR above the dewar 14 or may be installed to the inner wall via a mounting panel or the like. Similarly to FIG. 2A, as illustrated by the dashed-line box A, the first circuitry 21 may be placed on the floor surface in contact with or near the inner wall of the magnetically shielded room MSR.

With regard to the cables FLC connecting the first circuitry 21 and the second circuitry 22, the length of the cables FLC brought from the first circuitry 21 to the inner surface of the shielding material 90 is smaller than the length of the cables SQC connecting the SQUIDs 10 and the feedback coils 36 with the first circuitry 21. That is, the length of the cables FLC brought from the first circuitry 21 to the inner surface of the shielding material 90 is smaller than the length of the cables SQC brought from the dewar 14 to the first circuitry 21.

### Second Embodiment

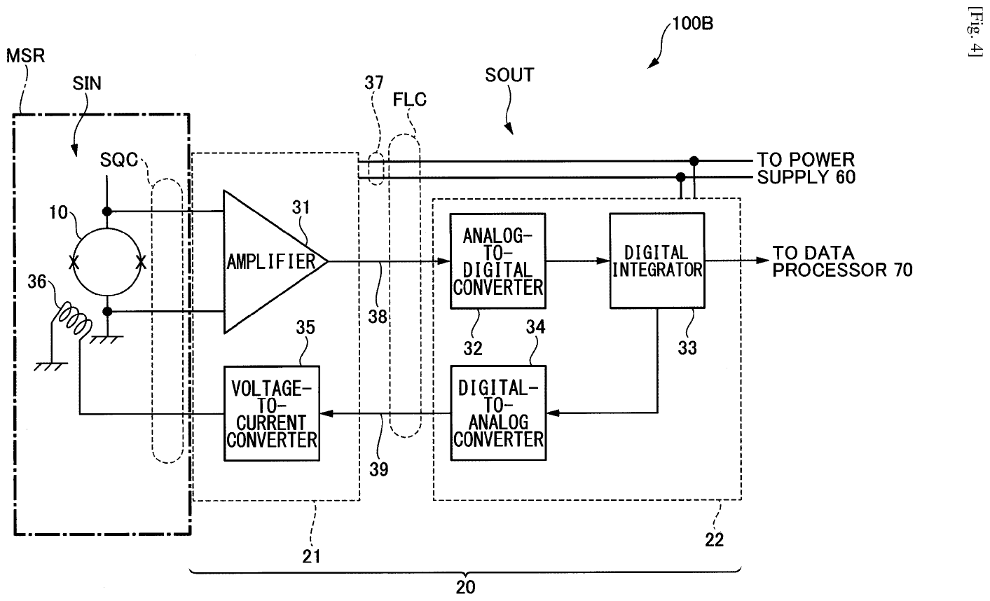

FIG. 4 is a block diagram illustrating an example of a magnetic-field measuring apparatus according to a second embodiment of the present invention. For elements similar to FIG. 1, the same numerals are used, and the detailed description will be omitted. The magnetic-field measuring apparatus 100B illustrated in FIG. 4 is similar to the magnetic-field measuring apparatus 100A illustrated in FIG. 1, except that the first circuitry 21 is installed outside the magnetically shielded room MSR.

That is, in the magnetic-field measuring apparatus 100B, the cables SQC pass through a through-hole formed in a wall of the magnetically shielded room MSR. For example, the magnetic-field measuring apparatus 100B uses a digital FLL system and may be applied to a magnetoencephalograph, a magnetospinograph, a magnetocardiograph, a magnetomyograph, or the like.

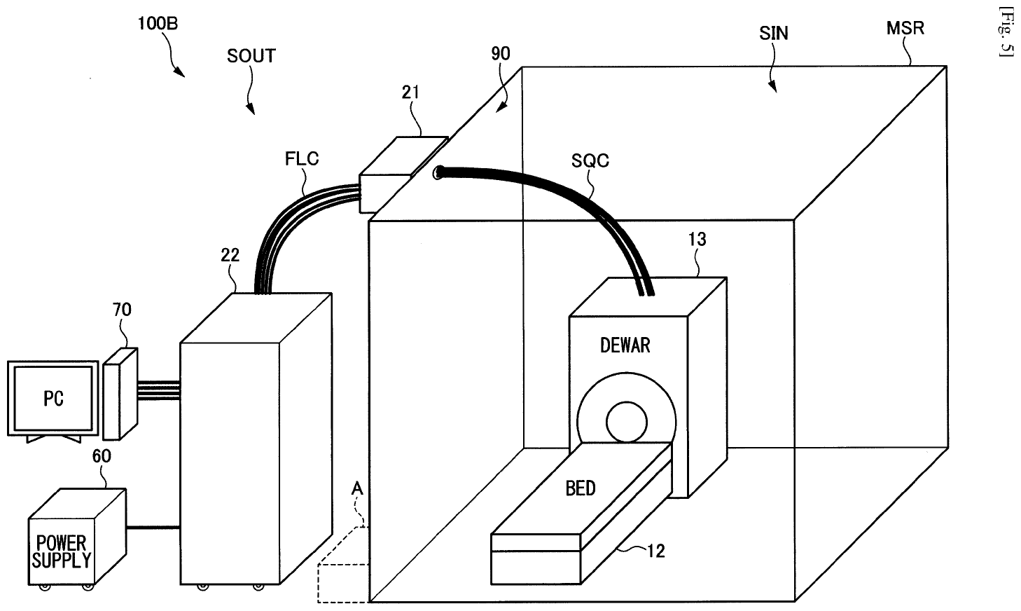

FIG. 5 is a layout diagram illustrating an example of installation of the magnetic-field measuring apparatus 100B of FIG. 4. For the same elements as the elements illustrated in FIG. 2A, the same numerals are used, and the detailed description will be omitted. The magnetic-field measuring apparatus 100B illustrated in FIG. 5 is a magnetoencephalograph similar to the magnetic-field measuring apparatus illustrated in FIG. 2A, and a bed 12 and a dewar 13 are disposed approximately at the center of the magnetically shielded room MSR.

In FIG. 5, the first circuitry 21 is installed to an outer wall (outer surface) obliquely above the dewar 13 of the shielding material 90 of the magnetically shielded room MSR, for example. That is, the first circuitry 21 is installed in contact with the outer wall of the shielding material 90 in the external space SOUT. The internal space SIN of the magnetically shielded room MSR can be effectively used by installing the first circuitry 21 outside the magnetically shielded room MSR. Similar to FIG. 2A, the cables SQC can be introduced to the cable insertion opening of the dewar 13 due to gravity, thereby reducing the influence of the load of the cables SQC applied to the cable insertion opening.

In this regard, the shielding material 90 may be protected by protection materials 91 as depicted in FIG. 2B. The protection materials may be gypsum boards, wooden boards, polyurethane foam materials, or the like. In such a case, instead of the first circuitry 21 being directly in contact with (the outer wall of) the shielding material 90, the first circuitry may be in contact with (the outer wall of) the shielding material 90 via the protection material 91. Such a variation in the relationship between the first circuitry 21 and the shielding material 90 may also apply to all of the other examples described herein.

The cables SQC from the dewar 13 are connected to the first circuitry 21 through a through hole formed obliquely above the dewar 13. The cables FLC connecting the first circuitry 21 and the second circuitry 22 are installed in the external space SOUT outside the magnetically shielded room MSR.

With regard to the cables SQC connecting the SQUIDs 10 and the feedback coils 36 with the first circuitry 21, the length of the cables SQC brought from the first circuitry 21 to the outer surface of the shielding material 90 is smaller than the length of the cables SQC brought from the SQUIDs 10 and the feedback coils 36 to the inner surface of the shielding material 90. That is, the length of the cables SQC brought from the first circuitry 21 to the outer surface of the shielding material 90 is smaller than the length of the cables SQC brought from the dewar 13 to the inner surface of the shielding material 90.

Similarly to FIG. 2A, the first circuitry 21 may be installed on a shelf 80 installed to the outer wall of the magnetically shielded room MSR. In this case, the external space SOUT around the magnetically shielded room MSR can be effectively used. Similarly to FIG. 2A, as illustrated by the dashed-line box A, the first circuitry 21 may be placed on the floor surface in contact with or near the outer wall.

Even in a case where the first circuitry 21 and the second circuitry 22 are thus installed outside the magnetically shielded room MSR, the first circuitry 21 and the second circuitry 22 are housed in separate housings. Therefore, in the external space SOUT, the degree of freedom of installing the first circuitry 21 and the second circuitry 22 can be increased.

For example, the first circuitry 21 may be placed in contact with or near an outer wall of the shielding material 90, regardless of the position where the second circuitry 22 is installed. The length of the cables SQC can be minimized when the first circuitry 21 is placed in contact with or near the outer wall. When the first circuitry 21 is placed in contact with the outer wall, the cables SQC are not exposed to the external space SOUT. This prevents the minute signals output from the SQUIDs 10 and transmitted through the cables SQC from being affected by radiant noise transmitted through the external space SOUT outside the magnetically shielded room MSR.

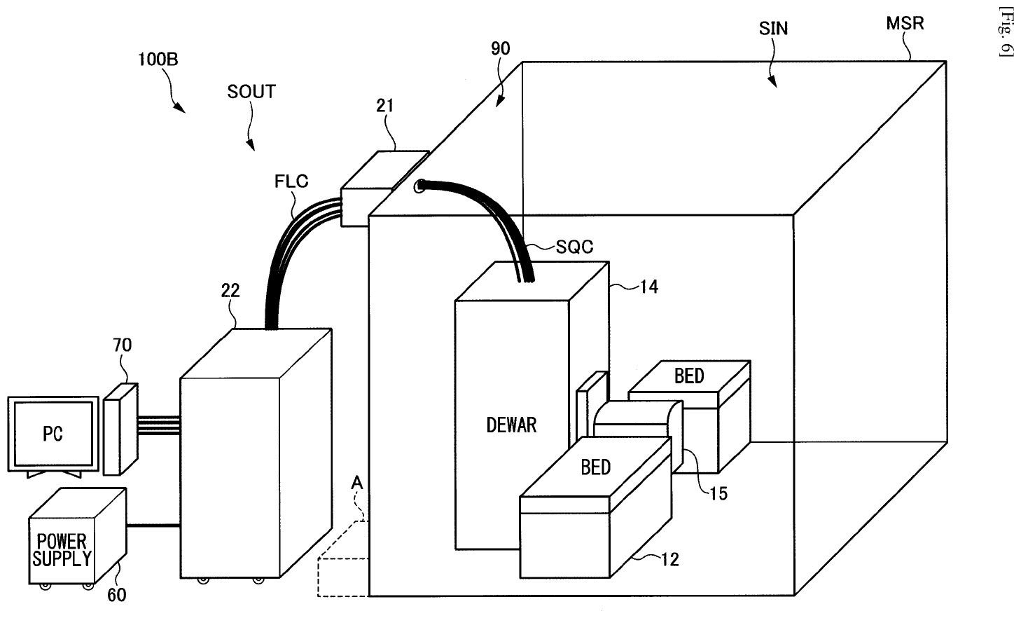

FIG. 6 is a layout diagram illustrating another example of installation of the magnetic-field measuring apparatus 100B of FIG. 4. For elements similar to FIG. 3, the same numerals will be used, and the detailed description will be omitted. For example, the magnetic-field measuring apparatus 100B illustrated in FIG. 6 is a magnetospinograph similar to the magnetic-field measuring apparatus illustrated in FIG. 3, and a split bed 12 and a case 15 of the SQUIDs 10 at a position corresponding to the patient's lower back are located approximately at the center of the magnetically shielded room MSR.

In FIG. 6, the first circuitry 21 is installed to an outer wall of the shielding material 90 of the magnetically shielded room MSR obliquely above the dewar 14, for example. The cables SQC installed from the dewar 14 are connected to the first circuitry 21 through a through hole provided in a wall obliquely above the dewar 14. The cables FLC connecting the first circuitry 21 and the second circuitry 22 are installed in the external space SOUT outside the magnetically shielded room MSR.

This allows for effective use of the internal space SIN of the magnetically shielded room MSR, similar to the examples of FIGS. 2 and 5. In addition, the cables SQC can be introduced to the cable insertion opening of the dewar 14 due to gravity, thereby reducing the influence of the load of the cables SQC applied to the cable insertion opening.

With regard to the cables SQC connecting the SQUIDs 10 and the feedback coils 36 with the first circuitry 21, the length of the cables SQC brought from the first circuitry 21 to the outer surface of the shielding material 90 is smaller than the length of the cables SQC brought from the SQUIDs 10 and the feedback coils 36 to the inner surface of the shielding material 90. That is, the length of the cables SQC brought from the first circuitry 21 to the outer surface of the shielding material 90 is smaller than the length of the cables SQC brought from the dewar 14 to the inner surface of the shielding material 90.

Similarly to the example of FIG. 3, as illustrated in the dashed-line box A, the first circuitry 21 may be placed on the floor surface in contact with or near the outer wall. In addition, similarly to the example of FIG. 5, when the first circuitry 21 and the second circuitry 22 are installed outside the magnetically shielded room MSR, the first circuitry 21 and the second circuitry 22 are housed in separate housings. Thereby, in the external space SOUT, the degree of freedom of installing the first circuitry 21 and the second circuitry 22 can be increased.

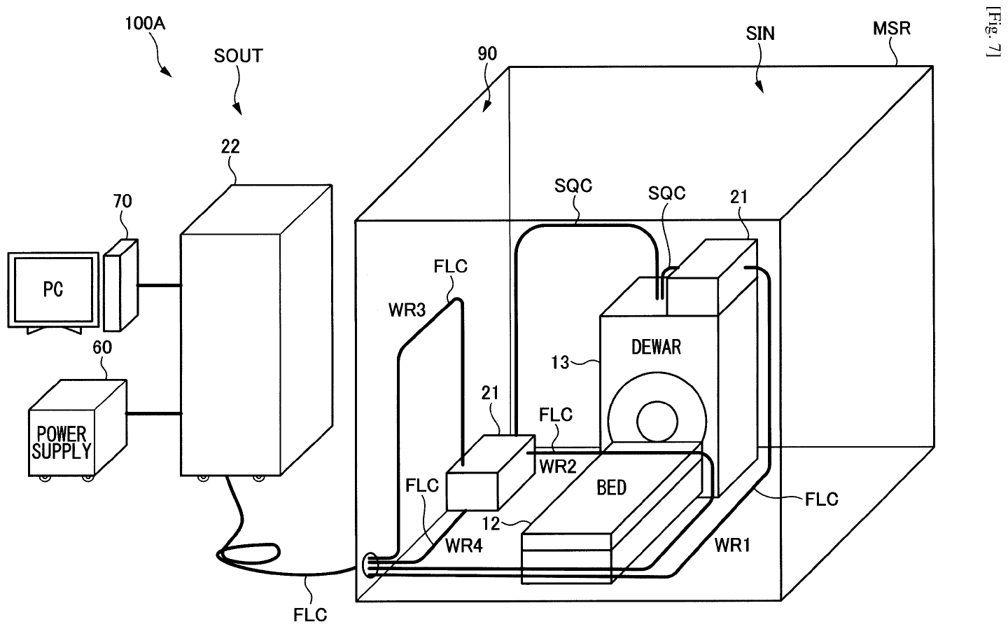

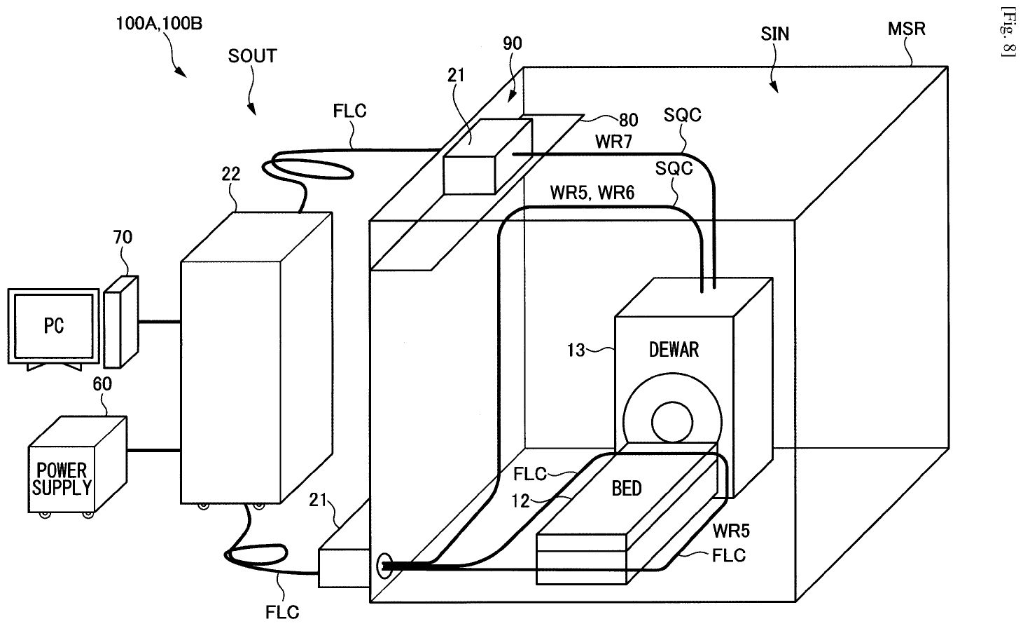

FIGS. 7 and 8 are explanatory diagrams illustrating examples of wiring routes in the magnetic-field measuring apparatus 100A of FIG. 1. The inventors installed the cables SQC and FLC to connect the dewar 13, the first circuitry 21, and the second circuitry 22 using seven wiring routes to investigate the influences of noise on the SQUIDs 10 depending on the difference in the wiring routes.

In a case of applying a wiring route WR1, the first circuitry 21 is placed on the top surface of the dewar 13 and the cables SQC are installed between the first circuitry 21 and the inside of the dewar 13. The cables FLC are brought installed downward from the first circuitry 21 to the floor along a side wall of the dewar 13 and then are installed around the bed 12. The cables FLC are then connected to the second circuitry 22 which is installed in the external space SOUT through a through hole, provided in a side wall of the magnetically shielded room MSR, near the floor. In the case of applying the wiring route WR1, the length of the cables SQC can be made smallest, but the length of the cables FLC in the magnetically shielded room MSR is great.

In the cases of applying wiring routes WR2, WR3, and WR4, the first circuitry 21 is placed on the floor surface in contact with or near the inner wall of the magnetically shielded room MSR (FIG. 7 illustrates an example in which the first circuitry 21 is in contact with the inner wall of the magnetically shielded room MSR). In the cases of applying the wiring routes WR2, WR3, and WR4, the cables SQC are installed upward along the inner wall of the magnetically shielded room MSR from the first circuitry 21 and then are connected to the SQUIDs inside the dewar 13. The length of the cables SQC in the cases of applying the wiring routes WR2, WR3, and WR4 is greater than the case of applying the wiring route WR1. For example, in each of the cases of applying the wiring routes WR2, WR3, and WR4, the length of the cables SQC is 10 meters, but the cables SQC are not particularly limited to the length.

In the case of applying the wiring route WR2, the cables FLC are installed from the first circuitry 21 over the bed 12 at a position facing the front surface of the dewar 13, and then are installed around the bed 12. The cables FLC are then connected to the second circuitry 22 which is installed in the external space SOUT through a through hole provided in a side wall of the magnetically shielded room MSR near the floor. In the case of applying the wiring route WR2, the length of the cables FLC is great as in the case of applying the wiring route WR1.

In the case of applying the wiring route WR3, the cables FLC are installed upward along the inner wall of the magnetically shielded room MSR from the first circuitry 21, then are installed downward along an inner wall to a through hole, pass through the through hole, and are connected to the second circuitry 22 which is installed in the external space SOUT. In the case of applying the wiring route WR3, the length of the cables FLC is great as in the cases of applying the wiring routes WR1 and WR2, but are installed away from the dewar 13.

In the case of applying the wiring route WR4, the cables FLC are installed on the floor surface from the first circuitry 21 up to a through hole, pass through the through hole, and are connected to the second circuitry 22 which is installed in the external space SOUT. In the case of applying the wiring route WR4, the length of the cables FLC can be minimized. For example, the length of the cables FLC in the case of applying the wiring route WR3 is 8 m, and the length of the cables FLC in the case of applying the wiring route WR4 is 1 m. For example, the size of the magnetically shielded room MSR is 2 m high by 3.6 m wide (at the wall surface having the through hole) by 2.3 m deep.

With reference to FIG. 8, in cases of applying wiring routes WR5 and WR6, the first circuitry 21 is placed on the floor surface in contact with or near an outer wall of the magnetically shielded room MSR. In the cases of applying the wiring routes WR5 and WR6, the cables SQC are brought into the magnetically shielded room MSR after passing through a through hole formed in a wall of the magnetically shielded room MSR facing the first circuitry 21. After being installed on the floor along the inner wall, the cables SQC are installed upward along the inner wall of the magnetically shielded room MSR and are brought into the dewar 13 from the top of the dewar 13.

In the case of applying the wiring route WR5, the cables FLC are brought from the first circuitry 21 into the internal space SIN through a through hole formed in a wall of the magnetically shielded room MSR near the floor. The cables FLC thus brought into the internal space SIN are installed around the bed 12, then, pass through the through hole again, and are installed up to the second circuitry 22 which is installed in the external space SOUT. In the case of applying the wiring route WR5, as in the case of applying the wiring route WR2 of FIG. 7, the cables FLC are installed over the bed 12 at a position facing the front surface of the dewar 13. The length of the cables FLC in the case of applying the wiring route WR5 is greater than the corresponding lengths in the other cases. In the case of applying the wiring route WR6, the cables FLC are not installed in the magnetically shielded room MSR, but are installed in the external space SOUT and are connected to the second circuitry 22.

In the case of applying the wiring route WR7, the first circuitry 21 is installed on a shelf 80 installed to an inner wall of the magnetically shielded room MSR obliquely above the dewar 13, similar to the case illustrated in FIG. 2A. In the case of applying the wiring route WR7, the cables SQC are brought downward from the first circuitry 21 into the dewar 13 and are connected to the inside of the dewar 13. Thus, in the case of applying the wiring route WR7, the length of the cables SQC is greater than the corresponding length in the case of applying the wiring route WR1, but is smaller than the corresponding lengths in the other cases of applying the wiring routes WR2-WR6 (e.g., the length of the cables SQC in the case of applying the wiring route WR7 is 2.2 m). In the case of applying the wiring route WR7, the cables FLC are not installed in the magnetically shielded room MSR, but are installed in the external space SOUT after passing through a through hole formed in a wall of the magnetically shielded room MSR, and are connected to the second circuitry 22.

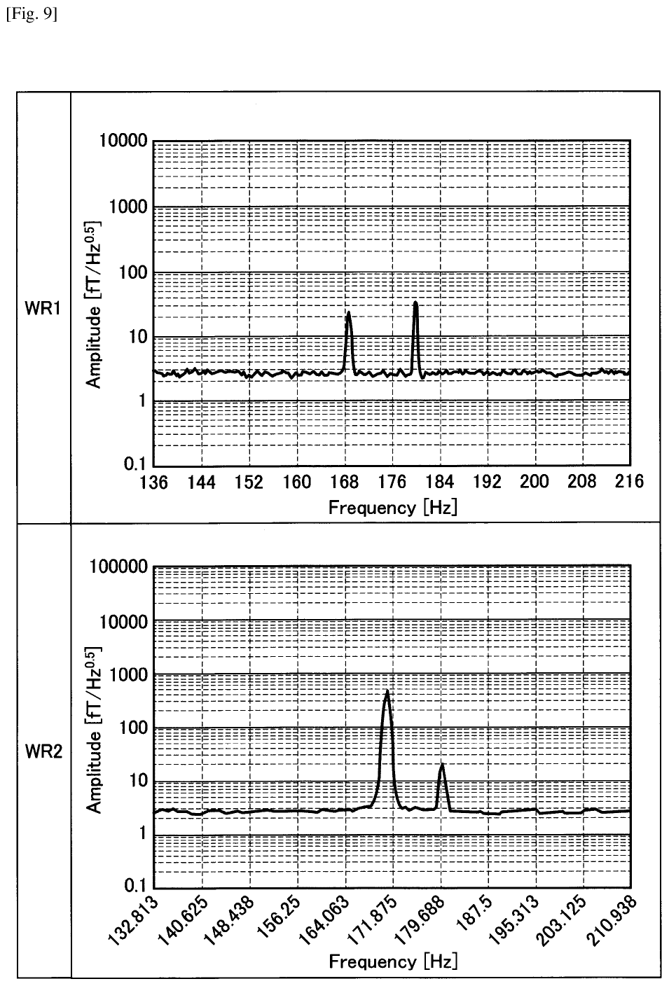

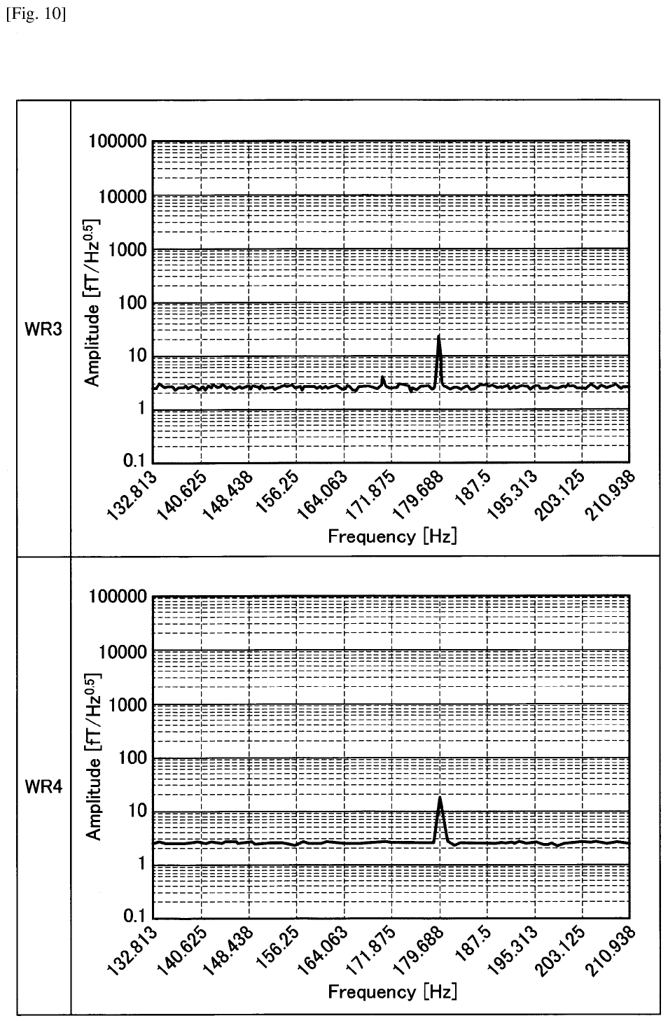

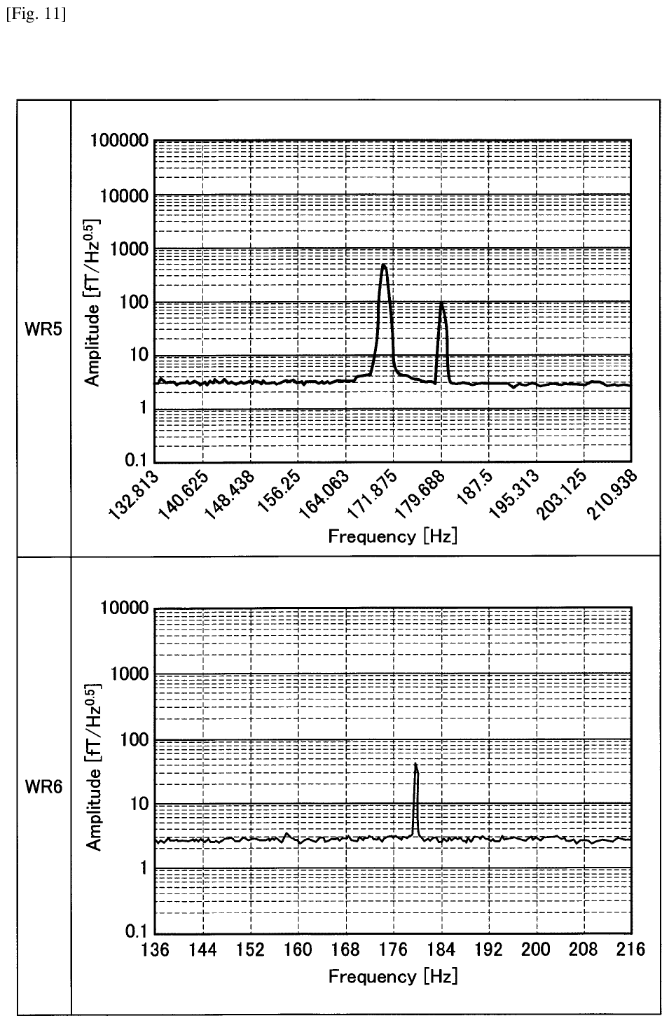

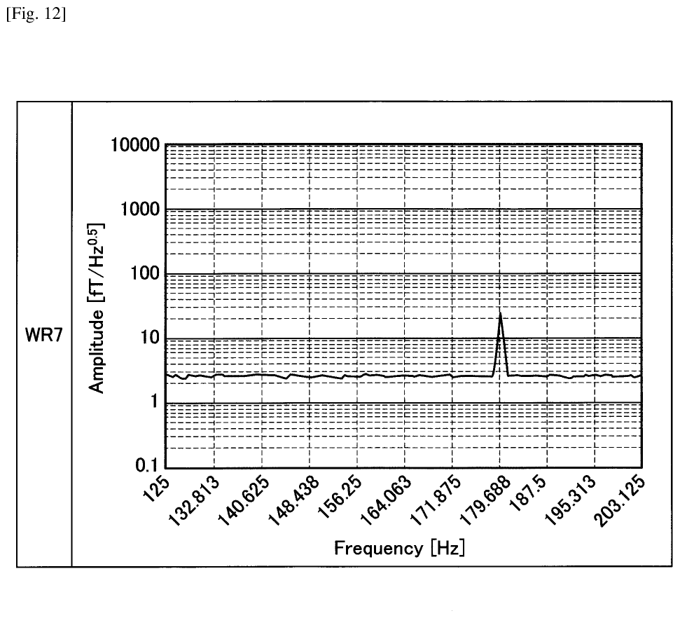

FIGS. 9-12 depict frequency spectrums for illustrating a measurement result of magnetic field noise in each case illustrated in FIGS. 7 and 8. FIG. 9 illustrates a measurement result of magnetic field noise in the cases of applying the wiring routes WR1 and WR2 illustrated in FIG. 7. FIG. 10 illustrates measurement results of magnetic field noise in the cases of applying the wiring route WR3 and WR4 illustrated in FIG. 7. FIG. 11 illustrates measurement results of magnetic field noise in the cases of applying the wiring routes WR5 and WR6 illustrated in FIG. 8. FIG. 12 illustrates a measurement result of magnetic field noise in the case of applying the wiring route WR7 illustrated in FIG. 8.

In the waveforms of FIGS. 9-12, 180 Hz magnetic field noise was constantly observed as a third harmonic noise of 60 Hz power supply noise. In the cases of wiring routes WR1, WR2, WR3, and WR5, magnetic field noise unrelated to power supply noise was observed around 168 Hz. The magnetic field noise around 168 Hz where the magnetic field noise was observed seems to be specific to the length of the cables FLC or the first circuitry 21, but the noise source is unknown.

In each of the cases of the wiring routes WR1, WR2, and WR5 where the cables FLC are installed near the dewar 13, magnetic field noise of 168 Hz was clearly observed. Also in the case of applying the wiring route WR3 where the cables FLC are installed for a long distance in the magnetically shielded room MSR, very small magnetic field noise of 168 Hz was observed.

On the other hand, magnetic field noise of 168 Hz was not observed in the case of applying the wiring route WR4 where the length of the cables FLC is small in the magnetically shielded room MSR and in each of the cases of applying the wiring routes WR6 and WR7 where the cables FLC are not installed in the magnetically shielded room MSR.

Thus, it can be seen that the nearer to the dewar 13 the cables FLC are installed, and the longer the cables FLC are installed in the magnetically shielded room MSR, the more likely the SQUIDs 10 detect magnetic field noise. That is, it can be seen that the positional relationships between the cables FLC and the dewar 13 correlate with magnetic field noise detected by the SQUIDs 10. It can be also seen that the length of the cables FLC in the magnetically shielded room MSR correlate with magnetic field noise detected by the SQUIDs 10.

For example, when the first circuitry 21 is positioned on the top surface of the dewar 13, as in the case of the wiring route WR1, the installation area of the first circuitry 21 can be made to overlap with the installation area of the dewar 13, and the inside of the magnetically shielded room MSR can be efficiently used. However, magnetic field noise is generated when the cables FLC are installed near the dewar 13 as a result of the first circuitry 21 being installed near the dewar 13. In this regard, if the dewar 13 is installed away from the cables FLC to avoid influence of magnetic field noise, the installation position of the dewar 13 becomes limited.

The minute signals output from the SQUIDs 10 through the cables SQC are not likely to be a source of noise because a change in amplitude is gentler than a change in amplitude of a digital signal. On the other hand, the length of the cables SQC is desirably as small as possible because the minute signals transmitted through the cables SQC are likely to be affected by radiation noise or the like.

Accordingly, it is desirable that the cables FLC are not installed in the magnetically shielded room MSR and that the length of the cables SQC be as small as possible in order to reduce magnetic field noise caused by the magnetic-field measuring apparatus 100A or 100B. To prevent the cables FLC from being installed in the magnetically shielded room MSR, it is desirable that the first circuitry 21 be located near or in contact with an inner or outer wall of the magnetically shielded room MSR.

In addition, when the first circuitry 21 is placed in the magnetically shielded room MSR, it is desirable that the first circuitry 21 is in contact with an inner wall of the magnetically shielded room MSR in order to prevent the cables FLC from being exposed to the inside of the magnetically shielded room MSR. When the first circuitry 21 is installed outside the magnetically shielded room MSR, the first circuitry 21 is desirably in contact with an outer wall of the magnetically shielded room MSR in order to prevent the cables SQC from being exposed to the external space SOUT.

For example, as a result of the installation position of the first circuitry 21 being determined in accordance with either one of the cases of the wiring routes WR4 and WR7 each satisfying these conditions, the magnetic-field measuring apparatus where magnetic field noise detected by the SQUIDs 10 can be minimized can be implemented. According to either one of the cases of applying the wiring routes WR4 and WR7, the length of the cables FLC (0 m or 1 m) brought from the first circuitry 21 to an inner wall (or a through hole) of the magnetically shielded room MSR is to be smaller than the length of the cables SQC (10 m), and consequently, the first circuitry 21 is to be placed near a wall of the sealing room.

Therefore, the measurement accuracy of biomagnetic field signals by the SQUIDs 10 can be improved. For example, by installing the first circuitry 21 at the position illustrated in any one of FIGS. 2, 3, 5 and 6, magnetic field noise detected by the SQUIDs 10 can be minimized.

### Third Embodiment

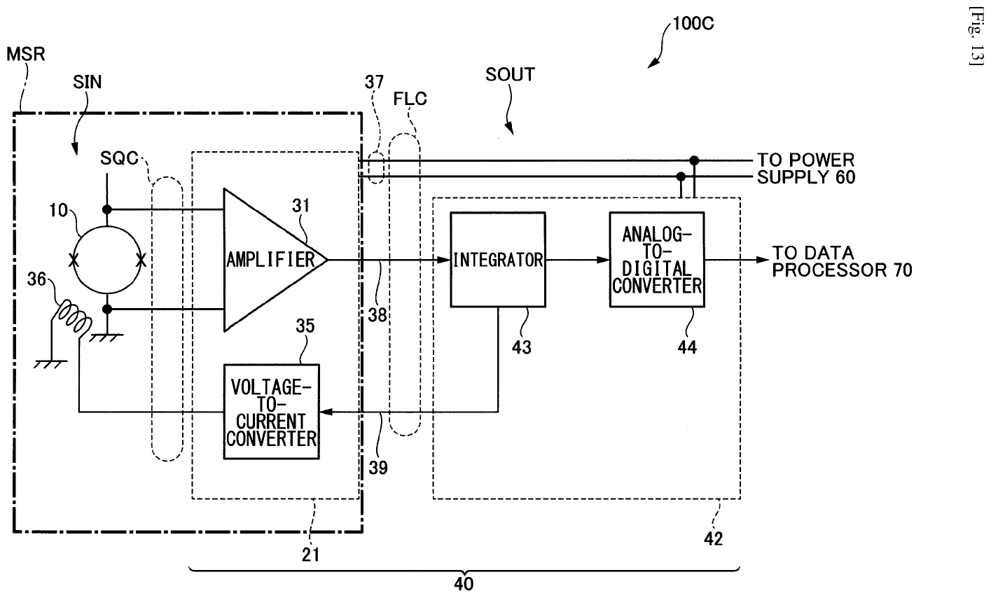

FIG. 13 is a block diagram illustrating an example of a magnetic-field measuring apparatus according to a third embodiment of the present invention. For elements similar to the elements illustrated in FIG. 1, the same numerals are used, and the detailed description will be omitted. The magnetic-field measuring apparatus 100C illustrated in FIG. 13 uses an analog FLL method and is applicable to a magnetoencephalograph, a magnetospinograph, a magnetocardiograph, a magnetomyograph, or the like.

The magnetic-field measuring apparatus 100C includes, for each channel, a SQUID 10, analog FLL circuitry 40, and a feedback coil 36. The analog FLL circuitry 40 includes, for each channel, an amplifier 31, an integrator 43, an analog-to-digital converter 44, and a voltage-to-current converter 35. The feedback coil 36 disposed near the SQUID 10 may be included in the analog FLL circuitry 40.

In the analog FLL circuitry 40, the amplifier 31 and the voltage-to-current converter 35 are included in the first circuitry 21 as in the example illustrated in FIG. 1. In the analog FLL circuitry 40, the integrator 43 and the analog-to-digital converter 44 are included in the second circuitry 42.

For example, the first circuitry 21 is disposed in the magnetically shielded room MSR together with the SQUID 10, and the second circuitry 42 is disposed outside the magnetically shielded room MSR. Similar to the example illustrated in FIG. 4, the first circuitry 21 may be installed outside the magnetically shielded room MSR together with the second circuitry 42.

The integrator 43 is analog circuitry and has a function similar to the function of the digital integrator 33 illustrated in FIG. 1. This allows the integrator 43 to integrate changes in voltage of the SQUID 10 (precisely, the amplified voltage output from the amplifier 31) from a working point of the Φ-V characteristic, and to output the integrated voltage (signal voltage) to the voltage-to-current converter 35 and the analog-to-digital converter 44. The analog-to-digital converter 44 converts the voltage from the integrator 43 to a digital value and outputs the digital value to the data processor 70.

In the magnetic-field measuring apparatus 100C, an installation example of the first circuitry 21 and the second circuitry 42 is the same as any one of the installation examples illustrated in FIGS. 2, 3, 5, and 6 described above. That is, the first circuitry 21 is disposed in contact with or near an inner or outer wall of the shielding material 90 of the magnetically shielded room MSR. The second circuitry 42 is installed in the external space SOUT external to the magnetically shielded room MSR.

This allows the magnetic-field measuring apparatus 100C of the present embodiment to reduce magnetic field noise generated in the magnetically shielded room MSR, similar to the above-described magnetic-field measuring apparatuses 100A and 100B, and prevents the SQUIDs 10 from detecting magnetic field noise. As a result, the measurement accuracy of biomagnetic field signals by the SQUIDs 10 can be improved.

In the above-described embodiments, the examples in which the first circuitry 21 is installed on an inner wall (inner surface) or an outer wall (outer surface) of the shielding material 90 of the magnetically shielded room MSR have been described, but the first circuitry 21 may be installed on a ceiling of the shielding material 90 of the magnetically shielded room MSR. In this case, the first circuitry 21 may be installed either inside (i.e., an inner surface of the shielding material 90) or outside (i.e., an outer surface of the shielding material 90) of the magnetically shielded room MSR.

Further, the first circuitry 21 may be located under the floor (i.e., an outer surface of the shielding material 90) when there is a space under the floor of the shielding material 90 of the magnetically shielded room MSR.

### Comparative Example 1

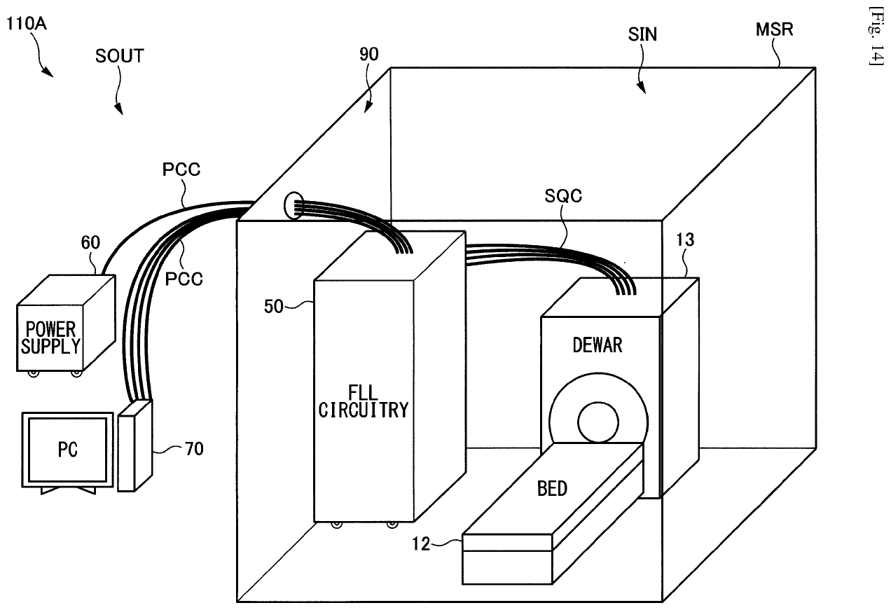

FIG. 14 is a layout diagram illustrating an example (comparative example) of installation of another magnetic-field measuring apparatus. For the same elements as the elements illustrated in FIG. 2A, the same numerals are used, and the detailed description will be omitted. The magnetic-field measuring apparatus 110A illustrated in FIG. 14 is a magnetoencephalograph, but may also be a magnetospinograph, a magnetocardiograph, a magnetomyograph, or the like. In the magnetic-field measuring apparatus 110A, FLL circuitry 50 is located inside the magnetically shielded room MSR.

For example, the FLL circuitry 50 is digital FLL circuitry and includes first circuitry 21 and second circuitry 22 illustrated in FIG. 1. Alternatively, the FLL circuitry 50 is analog FLL circuitry and includes first circuitry 21 and second circuitry 42 illustrated in FIG. 13.

The FLL circuitry 50 is connected to the dewar 13 via cables SQC. The FLL circuitry 50 is also connected to a power supply 60 and a data processor 70 which are located outside of the magnetically shielded room MSR via a plurality of electrical cables PCC including signal cables and power supply cables. Hereinafter, the electrical cables PCC will be simply referred to as cables PCC.

In the configuration illustrated in FIG. 14, because the FLL circuitry 50 is installed in the internal space SIN of the magnetically shielded room MSR, the SQUIDs 10 disposed in the dewar 13 may detect radiant magnetic field noise generated by the FLL circuitry 50 (especially the second circuitry 22 or 42). The SQUIDs 10 may also detect radiant magnetic field noise generated by the cables PCC because the cables PCC including the power supply cables supplying power to the FLL circuitry 50 are installed in the magnetically shielded room MSR.

On the other hand, the cables SQC connecting the SQUIDs 10 and the amplifiers 31 of the FLL circuitry 50 can be made to have a length equivalent to the length of the cables SQC illustrated in any one of FIGS. 2 and 3. Thus, the influence of noise on the minute signals transmitted through the cables SQC can be reduced.

In addition, when the FLL circuitry 50 is thus installed in the magnetically shielded room MSR, the available space in the internal space SIN becomes smaller. When the size of the magnetically shielded room MSR is increased accordingly, this increases the cost of a magnetic-field measuring system including the magnetic-field measuring apparatus 110A and the magnetically shielded room MSR.

### Comparative Example 2

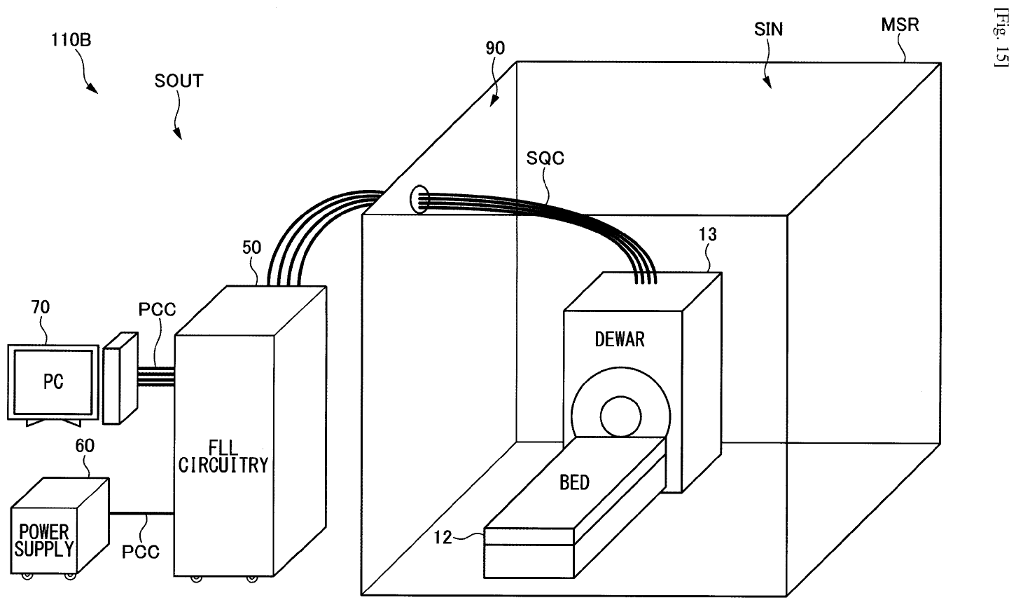

FIG. 15 is a layout diagram illustrating an example (comparative example) of installation of another magnetic-field measuring apparatus. For the same elements as the elements illustrated in any of FIGS. 2 and 14, the same numerals are used and the detailed description will be omitted. The magnetic-field measuring apparatus 110B illustrated in FIG. 15 is a magnetoencephalograph, but may also be a magnetospinograph, a magnetocardiograph, a magnetomyograph, or the like. In the magnetic-field measuring apparatus 110B, the FLL circuitry 50 is located outside of the magnetically shielded room MSR.

The cables SQC connected to the SQUIDs 10 (not illustrated) in the dewar 13 pass through a through hole formed in a wall of the magnetically shielded room MSR and are then connected to the FLL circuitry 50. The FLL circuitry 50 is connected to a power supply 60 and a data processor 70 located outside of the magnetically shielded room MSR, through a plurality of cables PCC including signal cables and power cables.

In the configuration illustrated in FIG. 15, because the FLL circuitry 50 is installed outside of the magnetically shielded room MSR, the SQUIDs 10 are not likely to detect radiant magnetic field noise generated from the FLL circuitry 50 or the cables PCC. On the other hand, because the cables SQC are installed up to the FLL circuitry 50 located outside the magnetically shielded room MSR, the minute signals transmitted to the cables SQC may be affected by noise. Because the FLL circuitry 50 is installed outside of the magnetically shielded room MSR, the internal space SIN of the magnetically shielded room MSR can be effectively used.

Although the magnetic-field measuring apparatuses have been described with reference to the embodiments, embodiments of the present invention are not limited to the above-described embodiments, and various modifications can be made within the scope of the present invention.

The present application is based on and claims priority to Japanese patent application No. 2019-182193 filed on Oct. 2, 2019 and Japanese patent application No. 2019-188211 filed on Oct. 11, 2019. The entire contents of Japanese priority application No. 2019-182193 and Japanese priority application No. 2019-188211 are hereby incorporated herein by reference.

### REFERENCE SIGNS LIST

10 SQUID
12 Bed
13, 14 Dewars
15 Case
20 Digital FLL circuitry
21 First circuitry
22 Second circuitry
31 Amplifier
32 Analog-to-digital converter
33 Digital integrator
34 Digital-to-analog converter
35 Voltage-to-current converter
36 Feedback coil
37 Power supply cable
38 Amplifier output line
39 Voltage line
40 Analog FLL circuitry
42 Second circuitry
43 Integrator
44 Analog-to-digital converter
50 FLL circuitry
60 Power supply
70 Data processor
80 Shelf
90 Shielding material
91 Protection material
100A, 100B, 100C Magnetic-field measuring apparatuses
110A, 110B Magnetic-field measuring apparatuses
FLC Electrical cable
MSR Magnetically shielded room
PCC Electrical cable
SIN Internal space
SOUT External space
SQC Electrical cable

### CITATION LIST

### Patent Literature

[PTL 1] Japanese Unexamined Patent Application Publication No. 11-118893

1. A magnetic-field measuring apparatus comprising:
a superconducting quantum interference device; and
flux-locked loop circuitry including first circuitry that includes an amplifier connected to an output of the superconducting quantum interference device, and second circuitry connected to the first circuitry,
wherein
the first circuitry is along an inner surface or an outer surface of a shielding material that separates an inside of a magnetically shielded room from an outside of the magnetically shielded room, the magnetically shielded room including the superconducting quantum interference device, and
the second circuitry is in the outside of the magnetically shielded room.

2. The magnetic-field measuring apparatus according to claim 1,
wherein
the flux-locked loop circuitry includes analog circuitry and digital circuitry,
the first circuitry includes only the analog circuitry, and
the digital circuitry is included in the second circuitry.

3. The magnetic-field measuring apparatus according to claim 1, wherein
a cable length from the first circuitry to the shielding material is smaller than a cable length between the superconducting quantum interference device and the first circuitry.

4. The magnetic-field measuring apparatus according to claim 1, wherein
the first circuitry is in contact with, directly or via another material, the inner surface or the outer surface of the shielding material.

5. The magnetic-field measuring apparatus according to claim 4, wherein
the first circuitry is installed at the inner surface or the outer surface of the shielding material.

6. The magnetic-field measuring apparatus according to claim 1, wherein
the first circuitry is on a shelf provided at the inner surface or the outer surface of the shielding material.

7. The magnetic-field measuring apparatus according to claim 1, wherein
the first circuitry is on a floor surface in the inside or the outside of the magnetically shielded room.

8. The magnetic-field measuring apparatus according to claim 1, further comprising:
a cryogenic container in which the superconducting quantum interference device is housed,
wherein
the first circuitry is on the inner surface or the outer surface of the shielding material and is higher than a top surface of the cryogenic container.

9. The magnetic-field measuring apparatus according to claim 1, wherein
a cable connecting the first circuitry with the second circuitry is at a position other than the inside of the magnetically shielded room.

10. The magnetic-field measuring apparatus according to claim 1, wherein
in a case where the first circuitry is along the outer surface of the shielding material, the first circuitry is housed in a housing and the second circuitry is housed in another housing.

11. The magnetic-field measuring apparatus according to claim 1, wherein
the second circuitry includes
an analog-to-digital converter configured to convert an amplified signal of the amplifier into a digital value,
an integrator configured to integrate converted digital values of the analog-to-digital converter, and
a digital-to-analog converter configured to convert an integrated value of the integrator into a voltage; and
the first circuitry further includes
a voltage-to-current converter configured to convert a converted voltage of the digital-to-analog converter into a current and to provide a converted current to a coil that is near the superconducting quantum interference device.

12. The magnetic-field measuring apparatus according to claim 1, wherein
the second circuitry includes
an integrator configured to integrate an amplified signal of the amplifier, and
an analog-to-digital converter configured to convert an integrated signal of the integrator into a digital value; and
the first circuitry further includes
a voltage-to-current converter configured to convert an integrated signal voltage of the integrator into a current and to provide a converted current to a coil that is near the superconducting quantum interference device.
